(19) United States
(12) Patent Application Publication
(10) Pub. No.: US 2014/0278489 A1
(43) Pub. Date: Sep. 18, 2014

(54) SYSTEMS AND METHODS FOR BROADCASTING APPOINTMENT AVAILABILITIES

(71) Applicant: ECLINICALWORKS, LLC, Westborough, MA (US)

(72) Inventors: Girish K. Navani, Shrewsbury, MA (US); Saurabh R. Singh, Newtown, MA (US); Harshavardhan D. Achrekar, Jersey City, NJ (US); Sivakumar K. Easwaran, Lowell, MA (US)

(21) Appl. No.: 14/198,125

(22) Filed: Mar. 5, 2014

Related U.S. Application Data
(60) Provisional application No. 61/789,072, filed on Mar. 15, 2013.
(60) Provisional application No. 61/870,363, filed on Aug. 27, 2013.

(51) Int. Cl. G06Q 10/06; G06Q 50/22

(52) U.S. Cl. CPC G06Q 10/06311; G06Q 50/22
USPC 705/2

(57) ABSTRACT

System and methods are disclosed for broadcasting notifications pertaining to appointment availabilities in schedules associated with medical practitioners. Medical practitioners are provided with access to a plurality of notification features. Selections may be received for activating the notification features and customizing settings for broadcasting notifications pertaining to the appointment availabilities. The settings can be used to identify a subset of patients that receive the notifications. The notifications are periodically transmitted to the subset of patients based on the selections provided by the medical practitioners. The notifications identify appointment availabilities in the schedules for the medical practitioners and permit the subset of patients to reserve the identified appointment availabilities. The subset of patients are provided with access to the notifications via the scheduling platform.

### COPYRIGHT NOTICE

A portion of the disclosure of this patent document contains material which is subject to copyright protection. The copyright owner has no objection to the facsimile reproduction by anyone of the patent document or the patent disclosure as it appears in the Patent and Trademark Office patent files or records, but otherwise reserves all copyright rights whatsoever.

### CROSS-REFERENCE TO RELATED APPLICATION

The present application claims priority to U.S. Provisional Patent Application No. 61/789,072 filed on Mar. 15, 2013 and U.S. Provisional Patent Application No. 61/870,363 filed on Aug. 27, 2013. The present application is also related to U.S. patent application Ser. No. ______ entitled “Systems and Methods for Broadcasting Appointment Availabilities” (Docket No. 0349205_trk1) which is being filed concurrently with the present application. All of these applications are incorporated by reference in their entirety.

### FIELD OF THE INVENTION

The present invention is directed to systems and methods for broadcasting appointment notifications and, more particularly, to identifying appointment availabilities in schedules associated with individuals or facilities (e.g., for healthcare services) and transmitting notifications to patients or customers to permit the appointment availabilities to be reserved.

### BACKGROUND OF THE INVENTION

Many industries and professions are dependent upon, or at least utilize to some extent, a schedule containing appointments or reservations. For example, in the medical field, schedules are routinely utilized to manage appointments that a medical practitioner has with his or her patients. Similarly, schedules are also typically utilized in the restaurant industry to manage dining reservations, and in the travel industry to manage transportation or lodging reservations (e.g., reservations for a plane, train, car, cruise or hotel). Schedules may also be utilized in the field of law to manage appointments that an attorney has with his or her clients, in the construction field to manage appointments that a contractor may have with different customers, in the government sector to keep track of appointments that a public servant may have with citizens, in the educational field to keep track of appointments that a teacher has with students, or in the entertainment industry to manage ticket reservations for a movie or show.

The manner in which a schedule is managed and maintained can have a major impact on a business (or governmental entity) in terms of both revenue generation and efficiency. For example, a business that is able to consistently fill all appointment availabilities in a schedule will be able to generate more revenue and serve a larger number of customers or patients. To help ensure that a schedule is consistently filled, a business or other entity should permit customers or patients to easily access a schedule, view available appointments or reservations, and reserve an appointment or make a reservation. Individuals who are employed by, or otherwise associated with, a business or entity for which the schedule is being maintained should also be permitted to easily make the schedules available to patients or customers, update the schedules and receive appointment or reservation requests from customers or patients. Unfortunately, many businesses or other entities suffer in terms of revenue generation and efficiency because they fail to provide these benefits.

One major problem that prevents businesses (or other entities) from fulfilling available appointments or reservations in a schedule is that patients or customers often cancel appointments or reservations after booking them. When a patient or customer cancels an appointment or reservation, it may be difficult to fill the appointment or reservation, especially if the cancellation occurs shortly before the time of appointment or reservation. This is due, at least in part, to the fact that the businesses are unable to quickly identify patients or customers who may be willing to fill the availabilities that have resulted from the cancellations and notify the identified patients or customers about the availabilities. Consequently, businesses that experience cancellations are unable to maximize the number of patients or customers that are serviced and are forced to forego profits that otherwise could have been realized.

Accordingly, there is a need for a platform that provides patients or customers with access to schedules and permits the patients or customers to quickly reserve availabilities in the schedules. There is also a need for such a platform to identify appointment availabilities in the schedules, select a subset of patients or customers who have a high probability of filling the appointment availabilities, transmit notifications to the identified subset of patients or customers to notify them of the appointment availabilities, and to permit the patients or customers to quickly and easily reserve the appointment availabilities. There is a further need for a such a platform to detect appointment availabilities in the schedules that have resulted from cancellations and to transmit notifications to patients or customers to fill the appointment availabilities.

### SUMMARY OF THE INVENTION

The disclosure herein relates to an appointment scheduling platform that permits the automatic transmission of notifications pertaining to appointment availabilities to patients or customers. Notifications may be transmitted for any opening in a schedule. In certain instances, the appointment availabilities may represent openings in a schedule that have resulted from cancelling appointments, rescheduling appointments or deleting appointments. The platform may be operated by or for a user such as an administrator or provider associated with a facility (e.g., a medical or other healthcare practice) that provides the services for which a patient or customer is interested in scheduling an appointment. A user can define one or more campaigns for broadcasting notifications. For each campaign, a user may select or enable a variety of different notification features that are configured to broadcast notifications in different scenarios. Interfaces may be displayed to the user to permit the user to identify the criteria that will trigger the platform to send the notifications. The user may customize the type of notification that is transmitted, the content of the notification and the subset of patients or customers that will receive the notification. The notification which identifies appointment availabilities may comprise an inbox notification, text message notification, e-mail notification, postal mail notification, automated phone call notification or other type of notification. The notification may include a function that permits a patient or customer to reserve one or more appointment availabilities.

A variety of notification features may be available to enable notifications to be transmitted in particular scenarios. The appointment scheduling platform allows users to customize various aspects of these features. A first notification feature relates to a wait list feature that transmits notifications pertaining to appointment availabilities to patients or customers included on a wait list. A second notification feature relates to a bump-up feature that transmits notifications pertaining to appointment availabilities to patients or customers who have already scheduled an appointment in order to permit the patients or customers to schedule an earlier appointment.

Additional notification features may transmit notifications to patients or customers after a previous visit to a medical facility. For example, a third notification feature may be provided which relates to an interval follow-up feature that identifies patients or customers who have not attended an appointment in a pre-determined time period or interval and transmits notifications to the identified patients or customers pertaining to the appointment availabilities. In certain embodiments, an individual may specify the time period or interval. The patients or customers who failed to attend or schedule an appointment in the time period may be sent notifications.

A fourth notification feature relates to a missed follow-up feature that transmits notifications to patients or customers that were asked to schedule a follow-up appointment after a prior visit to a facility, but failed to do so. In certain embodiments, a timeframe may be specified which indicates when patients or customers are or were expected to schedule a follow-up appointment and notifications may be transmitted if the patients or customers did not schedule a follow-up appointment within the timeframe. In certain embodiments, the patients or customers are only sent notifications relating to appointment availabilities that pertain to the same facility, practitioner or service provider that was associated with the prior visit.

In certain embodiments, the appointment scheduling platform may also include a subscriber notification feature that permits patients or customers to subscribe to or follow one or more practitioners, service providers or facilities. The subscriber notification feature can create a virtual community among users of the appointment scheduling platform in a manner which is similar to social networking sites (e.g., Twitter™). In certain embodiments, the subscriber notification feature may transmit notifications to patients or customers who are subscribers or followers of a practitioner, service provider or facility. The notifications may indicate appointment availabilities for the practitioner, service provider or facility. For example, if a patient or customer is a subscriber or follower of a medical practitioner who tends to have a schedule of appointments that is filled far in advance, the patient or customer can receive notifications when appointments become available as a result of subscribing to or following the medical practitioner. In certain embodiments, the practitioners, service providers or facilities may also be permitted to subscribe to or follow the patients or customers.

In certain embodiments, the subscriber notification feature may also transmit other types of notifications, such as notifications which indicate that the practitioner, service provider or facility has altered or updated profile information on the appointment scheduling platform (e.g., if the practitioner changed available days/hours, posted a message in a forum, provided a link to an article, updated a blog, changed offices or jobs, or provided other information via the appointment scheduling platform). In certain embodiments, the subscriber notification feature permits the medical practitioner to view all of the followers or subscribers of the medical practitioner. A user associated with the practitioner, service provider or facility can select an option that determines whether all subscribers or followers are to receive a notification or whether a particular subset of subscribers or followers is to receive a notification. The practitioner is provided a function for transmitting, posting or otherwise providing messages and information to followers or subscribers.

In accordance with certain embodiments, systems and methods are disclosed for broadcasting notifications pertaining to appointment availabilities in schedules associated with medical practitioners. A scheduling platform is adapted to transmit notifications identifying appointment availabilities in schedules for a plurality of medical practitioners. The scheduling platform may include a first portion that is accessible to the medical practitioners and a second portion that is accessible to a plurality of patients. Medical practitioners may be provided with access to a plurality of notification features via the second portion of the scheduling platform. Each of the notification features may be configured to transmit notifications to a subset of patients in a separate broadcast scenario and each is associated with broadcast configuration settings that permit the medical practitioners to customize the respective broadcast scenarios. A first set of selections may be received from the medical practitioners for activating one or more of the notification features. A second set of selections may be received from the medical practitioners for customizing broadcast configuration settings for each of the activated notification features. The broadcast configuration settings may be used to identify a subset of patients for receiving the notifications in accordance with the broadcast scenarios corresponding to the activated notification features. The notifications may be periodically (e.g., once or twice each day) transmitted to the subset of patients identified by the broadcast configuration settings. The notifications may identify appointment availabilities in the schedules for the medical practitioners and permit the subset of patients to reserve the identified appointment availabilities in the schedules. The subset of patients may be provided with access to the notifications via the first portion of the scheduling platform.

In accordance with certain embodiments, systems and methods are disclosed for broadcasting notifications pertaining to appointment availabilities in schedules associated with medical practitioners. A scheduling platform is adapted to transmit notifications identifying appointment availabilities in schedules for a plurality of medical practitioners. The scheduling platform includes a first portion that is accessible to the medical practitioners and a second portion that is accessible to a plurality of patients. Scheduling selections are received from patients via the first portion of the scheduling platform. The scheduling selections include selections for reserving appointment availabilities in the schedules, indicating appointment cancellations for existing appointments in the schedules, and enrolling on waitlists for one or more portions of the schedules of one or more of the medical practitioners. Broadcast selections are received from the medical practitioners for customizing broadcast configuration settings which at least identify a subset of patients for receiving the notifications. The broadcast configuration settings may comprise a look-up interval that specifies a timeframe for identifying patients having reserved appointments occurring in a pre-determined time period after an appointment cancellation. The subset of patients is selected to receive the notifications, at least in part, by (i) identifying patients who submitted wait list enrollment requests for portions of the schedules in which one or more appointment cancellations are received and (ii) identifying patients who reserved appointments falling within the timeframe specified by the look-up interval in response to one or more appointment cancellations. The notifications are periodically transmitted to the selected subset of patients. The selected subset of patients are provided with access to the notifications via the first portion of the scheduling platform. The notifications identify new appointment availabilities in the schedules of the medical practitioners that have resulted from the appointment cancellations and permit the selected subset of patients to reserve the new appointment availabilities.

These and other features and advantages will become apparent from the following detailed description of illustrative embodiments thereof, which is to be read in connection with the accompanying drawings.

### BRIEF DESCRIPTION OF DRAWINGS

The inventive principles are illustrated in the figures of the accompanying drawings which are meant to be exemplary and not limiting, in which like references are intended to refer to like or corresponding parts, and in which:

FIG. 1 is a block diagram of a system for broadcasting appointment availabilities in accordance with certain embodiments of the present invention.

FIG. 2 is a setup interface that permits a user to select broadcast times and medical practitioners for a broadcasting campaign in accordance with certain embodiments of the present invention.

FIG. 3 is an interface that includes a plurality of broadcast features that are selectable by a user for defining a broadcasting campaign that notifies patients of appointment availabilities in accordance with certain embodiments of the present invention.

FIG. 4A is an interface that includes broadcast configuration settings for the waitlist feature and the bump-up feature illustrated in FIG. 3 in accordance with certain embodiments of the present invention.

FIG. 4B is an interface that includes broadcast configuration settings for the interval follow-up feature illustrated in FIG. 3 in accordance with certain embodiments of the present invention.

FIG. 4C is an interface that includes broadcast configuration settings for the missed follow-up feature illustrated in FIG. 3 in accordance with certain embodiments of the present invention.

FIG. 4D is an interface that includes broadcast configuration settings for the subscriber feature illustrated in FIG. 3 in accordance with certain embodiments of the present invention.

FIG. 5 is an interface for defining rules for broadcasting notifications in accordance with certain embodiments of the present invention.

FIG. 6 illustrates an exemplary interface that permits a patient or customer to subscribe to or follow a medical practitioner or group of medical practitioners in accordance with certain embodiments of the present invention.

FIG. 7 is an illustration of a mobile device that is displaying an interface for scheduling appointments in accordance with certain embodiments of the present invention.

FIG. 8 is an illustration of a mobile device that is displaying an interface associated with a subscriber notification feature in accordance with certain embodiments of the present invention.

FIG. 9 is a flow chart of a method for broadcasting wait list notifications in accordance with certain embodiments of the present invention.

FIG. 10 is a flow chart of a method for broadcasting bump-up notifications in accordance with certain embodiments of the present invention.

FIG. 11 is a flow chart of a method for broadcasting interval follow-up notifications in accordance with certain embodiments of the present invention.

FIG. 12 is a flow chart of a method for broadcasting missed follow-up notifications in accordance with certain embodiments of the present invention.

FIG. 13 is a flow chart of a method for broadcasting subscriber notifications in accordance with certain embodiments of the present invention.

### DETAILED DESCRIPTION OF THE EMBODIMENTS

In the following description, reference is made to the accompanying drawings that form a part hereof, and in which is shown by way of illustration specific embodiments in which the invention may be practiced. It is to be understood that other embodiments may be utilized and structural changes may be made without departing from the scope of the present invention.

In accordance with certain embodiments, an appointment scheduling platform is disclosed that includes features for broadcasting appointment availabilities. The appointment scheduling platform may be utilized by individuals (e.g., doctors, other healthcare providers or individuals associated with such providers) that are seeking to fill available times slots in a schedule and individuals (e.g., patients or customers) that are seeking to schedule appointments. The appointment scheduling platform may be accessible through a network (e.g., the Internet) and may provide a variety of different features to facilitate the scheduling of appointments. Amongst other things, the appointment scheduling platform may permit the individuals who are seeking to fill available time slots in a schedule to create a schedule, or to otherwise provide access to a schedule, and to broadcast notifications pertaining to available time slots or appointments to the individuals that are seeking to schedule appointments. The individuals that are seeking to schedule appointments may access the appointment scheduling platform to search for available appointments, schedule appointments, cancel previously scheduled appointments and perform other types of related functions.

The individuals or facilities who are seeking to fill available time slots in a schedule by adding appointments may have an account with the appointment scheduling platform (e.g., a user account which is made available through a website and which may be accessed by providing a username, password or other login credentials). Upon accessing the account, the appointment scheduling platform provides appointment broadcast features that assist them in quickly and efficiently filling empty time slots in a schedule that have become available (e.g., possibly as a result of patients or customers cancelling one or more appointments). In certain embodiments, when a time slot in a schedule becomes available as a result of an appointment cancellation or for any other reason, the platform is able to broadcast notifications that include information about the newly available time slot to individuals that may be interested in booking or reserving the available time slot.

The manner in which appointment availabilities are broadcast can vary greatly. For example, in some cases, an individual who is seeking to schedule an appointment (e.g., a patient) may also have an account with the appointment scheduling platform. Thus, the appointment availabilities may be broadcast to individuals by transmitting notifications (e.g., messages, alerts or invitations) to inboxes, instant messaging features, or other communication features that are made available through the individual's account on the appointment scheduling platform. An appointment availability may also be broadcast by transmitting notifications via text messages, e-mails, letters (e.g., by postal mail service), automated phone calls, or in other ways.

In certain embodiments, the individuals seeking to broadcast appointment availabilities may access accounts provided by the appointment scheduling platform to customize the manner in which the appointment availabilities are broadcast. The appointment scheduling platform may provide these individuals with interfaces that permit the individuals to quickly customize the manner in which the appointment availabilities are broadcast. Amongst other things, the appointment scheduling platform may permit the individuals to specify who will receive notifications regarding the appointment availabilities (e.g., may specify that only a particular subset of patients or customers is to receive the notifications) and the manner in which the notifications are sent (e.g., may specify that notifications are broadcast by sending an inbox notification, e-mail or text message). The content, design and functionality of the notification may also be customized. For example, the individuals may select text, images, videos or hyperlinks to be included in the notifications and may configure the notifications to perform certain functions (e.g., the notifications may include options that permit a user to reserve an appointment, decline an appointment or cancel an appointment).

The discussion that follows describes an exemplary embodiment of the appointment scheduling platform that may be utilized in the medical field to facilitate the scheduling of appointments between medical practitioners and patients. However, it should be understood that the principles described herein are not limited to the health care field. Rather, the principles discussed herein may be applied to any situation in which individuals are seeking to book appointments or make reservations, and in which individuals or organizations are seeking to fill time slots in a schedule. For example, the principles discussed herein may be applied in the field of law (e.g., to book an appointment between a lawyer and a client), in the construction field (e.g., to book an appointment between a contractor and a customer), in the government sector (e.g., to book an appointment with a public servant and a citizen), in the educational field (e.g., to book an appointment between a teacher and a student), in the food industry (e.g., to book a reservation at a restaurant), in the travel industry (e.g., to book a ticket or reservation for a plane, train, car, cruise or hotel), in the entertainment industry (e.g., to book a ticket or reservation for a movie or show), or in any other field that involves booking appointments or reservations.

Embodiments described herein may be hardware-based, software-based and preferably comprise a mixture of both hardware and software elements. Thus, while the description herein may describe certain embodiments, features or components as being implemented in software or hardware, it should be recognized that any embodiment, feature or component that is described in the figures or description of the present application may be implemented in hardware and/or software. In certain embodiments, particular aspects are implemented in software, which includes but is not limited to firmware, resident software, microcode, etc.

Embodiments may include a computer program product accessible from a computer-usable or computer-readable medium providing program code for use by or in connection with a computer or any instruction execution system. A computer-usable or computer-readable medium may include any apparatus that stores, communicates, propagates or transports the program for use by or in connection with the instruction execution system, apparatus, or device. The medium can be a magnetic, optical, electronic, electromagnetic, infrared, or semiconductor system (or apparatus or device) or a propagation medium. The medium may include a computer-readable storage medium such as a semiconductor or solid state memory, magnetic tape, a removable computer diskette, a random access memory (RAM), a read-only memory (ROM), a rigid magnetic disk and an optical disk, etc.

A data processing system suitable for storing and/or executing program code may include at least one processor coupled directly or indirectly to memory elements through a system bus. The memory elements can include local memory employed during actual execution of the program code, bulk storage, and cache memories which provide temporary storage of at least some program code to reduce the number of times code is retrieved from bulk storage during execution. Input/output or I/O devices (including but not limited to keyboards, displays, pointing devices, etc.) may be coupled to the system either directly or through intervening I/O controllers.

Network adapters may also be coupled to the system to enable the data processing system to become coupled to other data processing systems or remote printers or storage devices through intervening private or public networks. Modems, cable modems and Ethernet cards are just a few of the currently available types of network adapters.

Referring now to the drawings in which like numerals represent the same or similar elements and initially to FIG. 1, a system 100 is disclosed for providing an appointment scheduling platform 140. As shown therein, a plurality of client devices 110 are in communication with a scheduling server 120 over a network 190. The network 190 may be any type of network such as one that includes the Internet, a local area network, a wide area network, an intranet, etc. The client devices 110, as well as the server 120, may be configured to communicate via wired or wireless communication links, or a combination of the two.

The client devices 110 may represent a desktop computer, laptop computer, cell phone, tablet device, or other type of computing device. Each of the client devices 110 may be equipped with one or more computer storage devices (e.g., RAM, ROM, PROM, SRAM, etc.) and one or more processing devices (e.g., a central processing unit) that are capable of executing computer program instructions. The one or more storage devices are preferably physical, non-transitory mediums. Any of the client devices 110 may further include a display that is capable of rendering graphical user interfaces and one or more input devices (e.g., keyboard, microphone, touch screen, camera, video camera, scanner, joystick, remote control device, etc.). Users (e.g., patients and medical practitioners) may utilize the input devices to interact with or manipulate the interfaces rendered on the display in order to communicate with the scheduling server 120.

As indicated in FIG. 1, some of the client devices 110 are being operated by patients 101, while other client devices 110 are located at a medical facility 160 (e.g., doctor's office, dental office, physical therapy office, hospital, or other type of location that is associated with a health care service) and may be operated by medical practitioners or employees that work at the medical facility 160. It should be noted that the terms “medical practitioner” and “medical provider,” as used herein, are meant to refer to any person that is associated with a medical practice or medical facility including, but not limited to, doctors, nurses, medical staff, and employees who work for or at the medical facility 160.

The scheduling server 120 may also include one or more processors and one or more computer storage devices. The one or more storage devices are preferably physical, non-transitory mediums. The scheduling server 120 may generally represent any type of computing device that is capable of communicating with a client device 110. In certain embodiments, the server 120 comprises one or more mainframe computing devices that execute a web server for communicating with client devices 110 over the Internet. The storage medium on the scheduling server 120 can store applications or software code for providing a scheduling platform 140 that is configured to assist medical practitioners with filling empty appointments and to assist patients 101 with scheduling appointments. Specifically, the scheduling platform 140 may be configured to provide these functionalities to medical practitioners and patients 101 by transmitting data for displaying interfaces on browsers that are executed by the client devices 110 which are being utilized by the medical practitioners and patients 101.

Both patients and medical practitioners may login (e.g., using a username, password or other login credentials) to an account on the scheduling platform 140. Different types of accounts and interfaces may be displayed based on whether a user is a patient or medical practitioner. For example, by logging into an account provided by a first portion of the scheduling platform, a medical practitioner or other user may be permitted to create, edit, delete or otherwise modify a centralized schedule 130 for a medical practitioner that may be searched by patients 101. A centralized schedule 130 may be created or updated using functions provided by the scheduling platform 140, or a user may choose to create or update a centralized schedule 130 by importing data from a local schedule 170 which is maintained on a client device 110 located at a medical facility 160. In certain embodiments, a medical practitioner may be permitted to sync a local schedule 170 on the client device 110 with a centralized schedule 130 stored on the scheduling server 120 such that changes made to the local schedule 170 (e.g., changes resulting from cancellations, new appointments, etc.) are automatically transmitted to the scheduling server 120 and utilized to update the corresponding schedule 130 maintained at the server 120, or vice versa.

As will be explained in further detail below, a user accessing an account associated with a medical practitioner may also be presented with interfaces associated with the appointment broadcast feature 150 that permit the user to create or modify notification broadcasting campaigns. The appointment broadcast feature 150 may permit the user to broadcast notifications about appointment availabilities (e.g., including availabilities that have become available due to appointment cancellations), and to further customize the manner in which the notifications are transmitted to patients 101. In certain embodiments, the appointment scheduling platform 140 may be configured to provide a “graphical wizard” that presents a series of interfaces to assist the user with customizing and sending notifications. For example, the graphical wizard may assist the user with identifying the patients that will receive the notifications, identifying triggers that will cause notifications to be sent, selecting the types of notifications that will be sent and customizing the content, design and functionality of the notifications. Any functions related to customizing or transmitting notifications to patients 101 may be performed by the appointment broadcast feature 150 that is provided as part of the scheduling platform 140.

On the other hand, by logging into an account associated with a patient 101, a user may be presented with interfaces that permit the user to search the centralized schedules 130 for available appointments, book appointments, cancel appointments, access communication features (e.g., an inbox or instant messaging function) and perform other related functions. As explained in further detail below, an account associated with a patient 101 may also permit the user to subscribe to or follow one or more medical practitioners or facilities and to select the type of notifications that the user would like to receive from the scheduling platform 140.

It should be noted that the system in FIG. 1 is merely meant to demonstrate an embodiment of an operating environment that can be utilized in conjunction with the principles taught herein, and should not be construed as limiting in any manner whatsoever. The particular configuration in FIG. 1 can be altered in numerous ways without departing from the principles described herein. For example, it should be noted that the functionality of the server 120 in FIG. 1 may be carried out by a plurality of servers. Likewise, although FIG. 1 depicts two medical facilities 160 and four client devices 110 which are in communication with the scheduling server 120, any number of medical facilities 160 and client devices 110 may be in communication with the scheduling server 120.

Furthermore, while FIG. 1 illustrates a plurality of client devices 110 in communication with a server 120 over a network 190, it should be recognized that the functionality provided by the scheduling server 140 (e.g., the functionally related to broadcasting appointment availabilities) may be performed by one or more client devices 110. For example, the client devices 110 associated with a medical practitioner may utilize an application and/or server that executes locally to communicate with other client devices 110 of the patients 101 and to perform the functions of the server 120. Thus, any functionality of the server 120 which is described herein can alternatively be implemented by a client device 110.

FIGS. 2, 3 and 4A-4D illustrate exemplary interfaces that may be utilized to configure notification broadcasting campaigns. A user associated with a medical practitioner account can establish one or more campaigns for broadcasting notifications to patients 101 pertaining to appointment availabilities. Each campaign can be customized to broadcast notifications for all or a subset of medical practitioners in a variety of different broadcasting scenarios.

A user seeking to establish a new campaign for broadcasting appointment availabilities for a medical practitioner or group of medical practitioners may initially be presented with the interface 200 in FIG. 2. The interface 200 includes a campaign name field 205 for specifying the name of the campaign. The interface 200 also includes features that permit a user to specify timeframes for sending notifications and for selecting one or more medical practitioners for whom the notifications will be sent.

More specifically, the top portion of the interface 200 includes a cancelled appointment option 201 and an open slot option 202 that are selectable by the user. The cancelled appointment option 201 permits a user to transmit notifications pertaining to appointments that have become available due to appointment cancellations. The open slot option 202 permits a user to specify that notifications pertaining to appointment availabilities are to be sent for other types of openings in a medical practitioner's schedule.

Upon activating the cancelled appointment option 201, the user can select specific types of cancellations that will trigger the sending of notifications to patients 101. For example, a cancellation type list 216 may be presented that includes options for different types of cancellations (e.g., cancellations received via phone calls or cancellations received via the scheduling platform 140 though user accounts associated with the patients 101). A user may specify that only certain types of cancellations should trigger the sending of notifications or that any cancellation should trigger the sending of notifications. The cancellation options selected by the user appear in the window 215 located to the right of the cancellation type list 216.

An interval selection feature 210 associated with the cancelled appointment option 201 permits a user to specify when notifications pertaining to availabilities that have resulted from cancellations are to be transmitted to the patients 101. A sliding bar or other input element may permit a user to determine when the notification is to be sent. In the exemplary interface 200 displayed in FIG. 2, the sliding bar is set to “2 weeks,” meaning that a notification will be sent two weeks prior to the date of the cancelled appointment (assuming the appointment has not been filled when the date arrives).

As an example, suppose that a patient 101 originally scheduled an appointment with a medical practitioner at 1 PM on Apr. 1, 2015, but decided to cancel the appointment on Feb. 1, 2015. Further suppose that the patient cancelled the appointment by making a phone call to the medical facility. In this situation, it is unlikely a medical practitioner would want to transmit a notification identifying the appointment availability on Feb. 1, 2015 since the date is still two months away. Thus, by adjusting the settings associated with the interval selection feature 210, the medical practitioner can specify that a notification informing patients 101 about the availability of the cancelled appointment should not be sent until two weeks before Apr. 1, 2015 (i.e., would not be sent until Mar. 18, 2015). More than one notification may be sent after this date. Moreover, since the patient cancelled the appointment by making a phone call, the scheduling platform 140 may perform a check before sending notifications to patients 101 to ensure that the user configuring the campaign selected an option for phone call cancellations from the cancellation type list 216. In certain embodiments, the scheduling platform may also determine whether a cancelled appointment was subsequently filled before sending notifications. In the case that a cancelled appointment was subsequently filled, a notification may not be sent.

The open slot option 202 permits a user to specify that notifications pertaining to appointment availabilities are to be sent for other openings in a medical practitioner's schedule (regardless of whether the appointment availabilities are related to cancellations). As mentioned above, each medical practitioner or group of medical practitioners may provide access to a schedule 130 via the scheduling platform 140. The appointment broadcast feature 150 may search the schedules 130 for availabilities and periodically transmit notifications to the patients 101. Like the cancelled appointment option 201, the open slot option 202 includes an interval selection feature 212 that specifies when notifications pertaining to appointment availabilities are to be transmitted to the patients 101. Once again, the sliding bar associated with the interval selection feature 212 is set to “2 weeks,” meaning that a notification will be sent two weeks prior to the date of the availability or opening in the schedule. More than one notification may be sent within the two-week period.

A user that has activated the open slot option 202 may further select an all slot option 213 or a rule-based option 214 to identify the appointment availabilities in the schedule that will be identified by the notifications. If the all slot option 213 is selected, notifications may be sent for any opening in the schedule. On the other hand, if the rule-based option 214 is selected, the appointment broadcast feature 150 may analyze certain rules that have been specified by the user to determine whether an opening in a schedule should be identified by or included in a notification. In certain embodiments, a medical practitioner or other user may be presented with a separate interface, overlay or pop-up window that permits rules to be defined.

For example, FIG. 5 discloses an exemplary interface 500 that provides a user with options 590A-590E for defining rules that may be utilized to determine whether an opening in a schedule should be identified by a notification when the rule-based option 214 is selected. A first option 590A permits the user to provide information indicating the availability of a practitioner or facility 160 for particular days and/or times. For example, this option may permit a user to indicate that a practitioner is generally available to take appointments from 9 AM to 5 PM on Tuesdays, Thursdays and Saturdays. Thus, notifications pertaining to appointment availabilities would not be sent for openings that fall outside of these days or times.

Another option 590B permits a user to create rules that indicate whether notifications should be sent only to existing patients 101 of a medical practitioner or facility 160 or, alternatively, to any patients 101 that have accounts with the scheduling platform 140. A third option 590C allows a user to indicate that notifications should only be sent to patients having particular types of medical conditions. For example, rules may be defined for a cardiologist which indicate that notifications should only be sent to patients 101 who have been diagnosed with heart-related conditions or who are seeking to schedule appointments for heart-related conditions. Additional options 590D and 590E permit rules to be defined based on the age and location of the patients 101. For example, rules may be defined that specify that notifications should only be sent to patients falling within a particular age range or located in a particular geographic area.

The interface 500 illustrated in FIG. 5 is meant to demonstrate exemplary categories of rules that may be utilized to customize the appointments that are identified by the notifications which are transmitted by the scheduling platform 140. However, it should be recognized that other types of rules may be defined by the user as well.

After the user has customized the cancelled appointment option 201 and/or open slot option 202, a provider selection feature 220 permits a user to specify that notifications are only to be sent for particular medical practitioners who are associated with a medical facility 160 or medical practice. For example, an account may be created on the scheduling platform 150 for a medical practice that includes multiple medical practitioners (e.g., doctors or professionals) and multiple medical facilities 160, but a user configuring a campaign for broadcasting notifications may wish to specify that notifications are only to be sent for some of the practitioners or facilities 150. Thus, the provider selection feature 220 allows a user to identify the practitioners and/or facilities for which notifications will or will not be sent.

In certain embodiments, the provider selection feature 220 may include a scrollable list 222 of practitioners. In order to identify a practitioner for which notifications will be sent, a user may select (e.g., by clicking with a mouse or by tapping a touch screen) a practitioner in the list 222 and select an add button 224. By selecting the add button 224, the name of the practitioner displayed in the list 222 will appear in the provider window 223 located on the bottom right portion of the interface 200. The provider window 223 will display all of the practitioners for which notifications will be sent. In order to remove a practitioner from the group for which notifications will be sent, the user may simply select a practitioner displayed in the provider window 223 and click on the remove button 225 located in the provider window 223.

A practitioner search feature 221 permits the user to search for a practitioner by name. A facility search feature 226 permits the user to narrow the list 222 of practitioners to only include practitioners from particular facilities. Other types of search features may be included to assist a user with rapidly identifying a medical practitioner.

When a user is done customizing the parameters included on the interface in FIG. 2, the user may select the “Save & Next” button located in the bottom right portion of the interface. The user may then be directed to another interface 300, e.g., as shown in FIG. 3, that permits the user to select and activate notification features. The exemplary interface 300 includes five notification features: a missed follow-up feature 301; wait list feature 302; bump-up feature 303; interval follow-up feature 304; and a subscriber notification feature 305. As explained in further detail below with respect to FIGS. 4A-4D, each of the notification features may be selected by the user to transmit notifications in a particular broadcast scenario and each may include broadcast configuration settings that may be tailored by the user to customize the broadcast scenarios.

Each notification feature includes a checkbox or other input element that permits a user to activate the notification features. Notifications will only be sent for the notification features that have been activated. The scheduling platform 140 will transmit notifications to patients for each notification feature that has been activated in accordance with the settings selected in FIG. 2. In certain embodiments, a notification feature that has been activated expands to present the user with additional broadcast configuration settings (as shown in FIGS. 4A-4D) that permit the user to further customize the manner in which notifications are transmitted.

The interface 400A in FIG. 4A illustrates exemplary configuration settings associated with a wait list notification feature 302 and a bump-up notification feature 303. The wait list notification feature 302 permits a user to send appointment availability notifications to patients 101 on a wait list. For example, a patient 101 may be enrolled or put on a wait list because a doctor has no available appointments during a particular time period. Despite the fact that the patient 101 desires to schedule an appointment immediately, the patient 101 may have to wait several days, weeks or even months before a doctor is able to see the patient 101. While the patient 101 may reluctantly accept an appointment that is several weeks away, the patient may also sign up for a wait list via the scheduling platform 140 (or may call a medical facility 160 to sign up). By signing up for the wait list, the patient 101 indicates that he or she is willing to accept an earlier appointment if one becomes available. Thus, by activating the wait list notification feature 302 (e.g., by selecting an activation button 330), a user may configure the scheduling platform 140 to transmit notifications that identify time slots associated with cancelled appointments or other appointment availabilities to patients 101 on a wait list.

The broadcast configuration settings associated with the wait list notification feature 302 may further permit the user to specify a subset of wait listed patients that are to be sent the notifications. For example, the wait list notification feature 302 may permit the user to specify that notifications are only to be sent to the subset of patients 101 who utilize a particular medical practitioner or group of medical practitioners identified by the provider selection feature 220 (e.g., by selecting button 331) or that notifications are to be sent to all wait listed patients 101 who established an account with the appointment scheduling platform 140 (e.g., by selecting button 332).

In certain embodiments, the wait list notification feature 302 may provide other options for narrowing the subset of wait listed patients 101 that will be sent notifications. For example, the wait list notification feature 302 may permit a user to specify that notifications are only to be sent to wait listed patients 101 who are seeking a particular visit type (e.g., only send notifications to patients seeking a routine check-up), patients that are seeking an appointment of a particular time length (e.g., only send notifications to patients seeking an appointment that is an hour in length), patients that are located in a particular geographic area, or patients that fall within a particular age range (e.g., only send notifications to patients between the ages of 50-65). Other types of narrowing options may also be presented.

The bump-up notification feature 303 located on the interface 400A performs a similar role. Specifically, the bump-up notification feature 303 allows notifications to be sent to patients 101 who have an appointment in the future in order to allow the patients 101 to schedule an earlier appointment (regardless of whether or not the patients 101 are on a wait list). A user may activate the bump-up notification feature 303 by selecting an activation button 340 on the interface 400A. The bump-up notification feature 303 may include a look-up interval feature 341 that permits notifications to be sent to patients who have future appointments falling within a particular timeframe that is identified by the look-up interval feature 341 or which occurs after a date identified by look-up interval feature 341.

Users may identify a particular subset of patients 101 that are to receive notifications using the set interval feature 342. Specifically, in addition to narrowing the subset of patients 101 based on the criteria specified by the look-up interval feature 341, the set interval feature 342 may permit a user to further narrow the subset of patients 101 to consist of patients having appointments with the same visit duration (e.g., an hour) as a cancelled appointment or to consist of patients 101 having an appointment with the same type of visit (e.g., a visit associated with a routine check-up) as a cancelled appointment. In certain embodiments, additional types of narrowing criteria may also be selected by the user (e.g., based on the location of patient, age of patient, etc.).

FIG. 4B illustrates another exemplary interface 400B that includes an interval follow-up feature 304. A user may activate the interval follow-up feature 304 by selecting an activation button 410 on the interface 400B. Generally speaking, a follow-up appointment refers to an appointment that is scheduled after an initial medical visit. A patient may be asked to schedule a follow-up appointment with a medical practitioner for a variety of different reasons (e.g., to ensure that the patient is healing properly, to ensure that medication is working properly or to monitor a medical condition). The interval follow-up feature 304 can be utilized to send notifications to patients after a pre-determined period of time has expired since their last medical visit. In certain embodiments, this feature may be particularly useful for scenarios where medical practitioners desire to transmit notifications about appointment availabilities to patients who are expected to frequently attend medical visits (e.g., elderly patients or patients having chronic illnesses).

In response to detecting an appointment cancellation (e.g., in the case that cancelled appointment option 201 is selected) or other appointment availability (e.g., in the case that open slot option 202 is selected), the interval follow-up feature 304 may identify patients that have not attended, or at least scheduled, a follow-up visit in a pre-determined period of time. The interval follow-up feature 304 may then transmit notifications to the identified patients 101 to notify the patients 101 about the availabilities. For example, after a patient 101 visits a medical practitioner for an annual check-up or for a particular condition (e.g., the patient has the flu), the patient 101 is often expected to schedule a subsequent visit with the doctor (e.g., for another check-up after a year has passed or to make sure medicine prescribed to the patient is working). However, the patient 101 may fail to schedule or attend a subsequent visit. In some cases, extended periods of time may elapse (e.g., a year or two). Thus, the interval follow-up feature 304 may identify appointment availabilities and transmit notifications to these patients to notify the patients that appointments are available. Patients 101 who receive the notifications may reserve an appointment for a follow-up visit. Like the other notification features, the interval follow-up feature 304 transmits the notifications to the identified patients in accordance with the configuration settings specified in FIG. 2.

The interval follow-up feature 304 includes a variety of settings that permit a user to define the subset of patients who will receive the notification. For example, interval follow-up feature 304 may include a set interval feature 420 that permits a user to specify a timeframe or interval. As explained in further detail below, an age group feature 430 and diagnosis feature 440 further permit the user to define the subset of patients based on age and diagnosed medical condition. Patients who have not scheduled or attended an appointment with a medical practitioner or a particular medical facility 160 during the timeframe identified by the interval follow-up feature 304 may be sent notifications after the timeframe has expired. For example, if the set interval feature 420 identified a two-month timeframe or interval, then the interval follow-up feature 304 would identify patients who did not attend a follow-up visit in the previous two months and transmit notifications to the identified patients 101 (at the time specified by the interval selection features 210 and 212).

To determine whether a patient 101 attended an appointment within the timeframe specified by the set interval feature 420, the scheduling platform may analyze an appointment calendar associated with a patient's account on the scheduling platform 140 and/or may analyze practitioner schedules 130 associated with practitioner accounts. A user may select provider options 425 and 426 to specify whether the interval follow-up feature 304 should only account for appointments that were scheduled for the practitioners associated with the user account (e.g., the practitioners identified by provider selection feature 220) or whether appointments associated with any practitioner should be taken into consideration.

If a first provider option 425 is selected, the interval follow-up feature 304 will only consider appointments for the practitioners associated with the account. Notifications may be sent to the patients 101 who failed to schedule appointments with these practitioners within the specified timeframe. Alternatively, if a second provider option 426 is selected, the interval follow-up feature 304 will consider whether the patient scheduled an appointment with any practitioner within the specified timeframe. Notifications will be sent if the patient has failed to schedule an appointment with any practitioner within the identified timeframe.

A user may further restrict the sending of notifications to include patients of a particular age group and/or patients having a particular diagnosis. For example, the age group feature 430 permits a user to specify that notifications are only to be sent to follow-up patients within a particular age range specified by the age group feature 430. The interval follow-up feature 304 may further include a diagnosis feature 440 that permits a user to specify that notifications should only be sent to patients 101 that have been diagnosed with a particular illness or condition. In certain embodiments, the diagnosis feature 440 may present a user with a diagnosis search feature 450 that permits the user to search for a particular diagnosis by entering text queries into a text field, as well as a scrollable list 460 of diagnoses. The user may select a diagnosis using the diagnostic search feature 450 and/or scrollable list 360. Any follow-up patients 101 associated with a selected diagnosis may be sent notifications relating to cancelled appointments or other openings which have become available.

Similar to the provider selection feature 220 described above, a user may select (e.g., by clicking with a mouse or by tapping a touch screen) a diagnosis in the list 460 and select an add button 470 to include the diagnosis in the diagnosis window 480. By selecting the add button 470, the name of the diagnosis, or the ICD code associated with the diagnosis, will be displayed in the diagnosis window 480 located on the right portion of the interface 400. The diagnosis window 480 displays all of the diagnoses associated with follow-up patients for which notifications will be sent (note: the exemplary diagnosis window 480 illustrated in FIG. 4B is empty because none of the diagnoses have been selected). In order to remove a diagnosis from the diagnosis window 480, the user may simply select a diagnosis shown in the window 280 and select the remove button 475.

FIG. 4C illustrates an interface 400C that includes a missed follow-up feature 301 that may be utilized to transmit notifications to patients 101 who have missed or failed to schedule follow-up appointments for medical visits at a medical facility 160 or with a medical practitioner. A user can activate and configure the missed follow-up feature 301 in order to transmit notifications to patients who were expected to schedule or attend a follow-up visit, but who failed to do so. In certain embodiments, the notifications may only include information relating to appointments that are available at the same medical facility 160 or for the same medical practitioner as the initial or prior visit. The notifications may be transmitted in accordance with the selections made in FIG. 2. Like any other notification described herein, the notifications may permit the patients 101 to reserve or book one or more of the appointments identified in the notification.

The missed follow-up feature 301 may include an activation button 501 for initially activating the missed follow-up feature 301. In certain embodiments, the missed follow-up feature 301 may also include a timeframe feature 520 that permits a user to specify a period of time indicating when patients are or were expected to schedule a follow-up appointment. In certain embodiments, the timeframe feature 520 may include two input elements: a first “look back” input element that permits the user to specify a starting date for the timeframe, and a “look ahead” input element that permits a user to specify an ending date for the timeframe. Notifications may be transmitted to the patients who failed to schedule a follow-up appointment within the timeframe or interval defined by the starting date and the ending date. For example, in the exemplary interface 400C shown in FIG. 4C, the look back input element is set to four days in the past and the look ahead input element is set to six days in the future, thus defining a timeframe or interval of ten days. Therefore, notifications pertaining to appointment availabilities may be transmitted to any patients 101 who were expected to schedule an appointment within the past four days but who failed to do so, or who are expected to schedule an appointment within the next six days and have not yet done so.

The notifications pertaining to appointment availabilities may be sent in response to the occurrence of certain events. In certain embodiments, a patient 101 is given a predetermined period of time to schedule an appointment after a visit to a medical facility 160. For example, if a patient 101 fails to schedule an appointment within a certain period of time, the missed follow-up feature 301 may automatically transmit a notification to the patient. In certain embodiments, the missed follow-up feature 301 may track information that indicates when a patient 101 has visited a medical facility 160. An automatic check may be performed after the expiration of a certain period of time (e.g., 14 days) to determine whether the patient scheduled a follow-up appointment. In certain embodiments, the period of time may vary based on the reason for a medical visit (e.g., the check may be performed in seven days if the user had the flu or one year if the user went for a routine annual check-up). In certain embodiments, a medical practitioner or other user can specify the period of time. Regardless of how the period of time is defined, notifications may be sent to the patient 101 if it is determined that the patient has not scheduled a follow-up appointment.

In certain embodiments, the sending of the notification may also be triggered in response to appointment cancellations. For example, each time an appointment is cancelled, the missed follow-up feature 301 may identify all patients that were or are expected to schedule an appointment within the time period specified by the timeframe feature 301. For the patients 101 who failed to schedule an appointment within the specified time period, a notification may be transmitted to the identified patients. Other types of events may trigger the sending of notifications as well (e.g., a medical practitioner can manually initiate the sending of a notification).

The missed follow-up feature 301 also includes a set of provider options 525 and 526 that operate in a similar manner as the provider options 425 and 426 for the interval follow-up feature. If the first provider option 525 is selected, it will be assumed that a patient missed a follow-up visit if the patient did not attend an appointment with the medical practitioner who provided medical services to the patient during an initial or prior visit (even if the patient attended a medical visit with a different medical practitioner or came in for an appointment at the same medical facility and was administered services by a nurse or physician's assistant). However, if the second provider option 526 is selected, any appointment attended by the patient will be accounted for and it will be assumed that a patient missed a follow-up visit if the patient did not attend a medical visit with any medical practitioner.

The interface 400D in FIG. 4D also includes a subscriber notification feature 305 that permits medical practitioners to send notifications to patients 101 who are subscribed to, or who are following, a medical practitioner or group of medical practitioners in accordance with certain embodiments of the present invention. As explained in further detail below with respect to FIG. 6, patients 101 may indicate that they want to receive notifications pertaining to appointment availabilities (or other types of notifications) for particular medical practitioners and/or medical facilities 160. In addition to receiving notifications pertaining to appointment availabilities, a patient 101 may receive other types of notifications that are relevant to a medical practitioner including notifications that pertain to messages or articles posted by a medical practitioner (e.g., in a forum or on a blog associated with the scheduling platform 140) or changes made to a profile associated with the medical practitioner. For example, a medical practitioner may update a profile on the appointment scheduling platform 140 in order to adjust the practitioner's available days or hours, indicate a change in employment or office location, or for other reasons. The subscriber notification feature 305 may transmit notifications to subscribers or followers of the medical practitioner in response to making such updates.

A medical practitioner or other user may activate the subscriber notification feature 305 by selecting an activation button 531. In certain embodiments, the subscriber notification feature 305 may include a practitioner window 532 that lists one or more medical practitioners associated with a particular user account for the appointment scheduling platform 140. The practitioner window 532 may also include entries which represent groupings of the medical practitioners listed in the practitioner window 532 (e.g., “All Providers”). In certain embodiments, the practitioner window 532 may be utilized to enable and/or prevent subscriber notifications from being broadcast for the practitioners listed in the practitioner window 532. Next to the name of each practitioner, or group of practitioners, is an indicator 534 that displays the number of subscribers or followers for each medical practitioner or group of medical practitioners. A subscriber window 533 may display the names of the subscribers or followers of a selected entry in the practitioner window 532. In certain embodiments, the subscriber window 533 may be utilized to enable and/or prevent subscribers from receiving notifications pertaining to one or more of the practitioners listed in the practitioner window 532.

FIG. 6 illustrates an interface 600 associated with a subscriber notification feature that permits patients 101 to subscribe to or follow a medical practitioner or group of medical practitioners in accordance with certain embodiments of the present invention. The exemplary interface 600 shown in FIG. 6 may be displayed to users who access accounts on the appointment scheduling platform 140 that are associated with patients 101.

In certain embodiments, the patient 101 may be presented with a provider list window 610 that includes the names of medical practitioners (e.g., “John Smith, MD” or “Jane Doe, DO”) or medical facilities (e.g., “Mercy Medical Hospital”). The provider list window 610 may display a scrollable list of the medical practitioners and medical facilities. A patient can browse the list in the provider list window 610 and identify medical practitioners or medical facilities that the patient 101 wishes to subscribe to or follow. In certain embodiments, a set of controls 614 may be provided that permit a patient 101 to filter or sort the list of medical practitioners. For example, the controls 614 may be used to filter the list of medical practitioners based on geographic location, practice area or other criteria. Likewise, the controls may also permit the user to sort the list of medical practitioners based on ratings (e.g., ratings or feedback provided by patients or other medical practitioners), earliest availability, experience or other criteria.

In order to follow or subscribe to a medical practitioner listed in the provider list window 610, a patient 101 may select the name of a medical practitioner (e.g., by clicking on the name with a mouse or by performing a gesture on the name) and subsequently select the add button 611. Upon selecting the add button 611, the name of the medical practitioner may appear in the subscriber window 620, which lists all of the medical practitioners and medical facilities that the patient has subscribed to or is following. A patient 101 may indicate that he or she no longer wishes to follow a medical practitioner by selecting the practitioner in the subscriber window 620 and selecting the remove button 612.

A patient 101 is not limited to subscribing to or following individual medical practitioners. In certain embodiments, medical practitioners may be included in groups and a patient 101 may be permitted to follow or subscribe to a group of practitioners. A patient 101 may add or remove groups of medical practitioners to or from the provider list window 610 and subscriber window 620 in the same manner discussed above. The exemplary interface 600 depicted in FIG. 6 illustrates a variety of groups that may be followed, or subscribed to, by patients 101 (e.g., “Mercy Medical Hospital,” “Podiatrists in New York City,” “South Nassau Hospital,” “Optometrists in Long Beach, N.Y.” and “Custom Group”). The patient 101 that has subscribed to or is following a group may be sent notifications relating to any of the practitioners included in the group. The groups may be created in various ways or based on various factors. For example, practitioners may be grouped according to medical facility or practice area (e.g., dermatology, pediatrics, radiology, or other practice area). Practitioners may also be grouped based on geographic location, experience, feedback or ratings provided by patients, practitioners and/or co-workers, availability, price (e.g., how expensive the medical practitioner is relative to other practitioners) or any combination of the above parameters and other parameters.

In certain embodiments, groups of practitioners may be created by the subscriber notification feature or other component of the appointment scheduling platform 140. In certain embodiments, a patient 101 may define a new group by selecting a group creation button 613 on the interface 600. After selecting the group creation button 613, the patient may be presented with a form that permits the patient 101 to create a custom group by selecting various parameters to tailor the list of providers that are included in the group. For example, a patient 101 may define a custom group of practitioners by selecting a geographic area (e.g., “New York City, N.Y.”), practice area (e.g., dermatology), and user rating (e.g., average rating above 3 on a scale of 1-5). Any medical practitioner that satisfies the criteria defined by the patient 101 may be included in a new group and the patient 101 can specify a name for the group.

The interface 600 associated with the subscriber notification feature 601 may also include a notification selector feature 630 that permits a patient 101 to identify or select the notifications that should be transmitted to the patient. For example, in FIG. 6, the patient 101 has opted to receive “All” types of notifications. However, the patient 101 has the option of selecting particular types of notifications if desired (e.g., the patient 101 may indicate that he or she only wishes to receive notifications related to appointment availabilities).

As with any other notification feature described in this disclosure, the subscriber notification feature 305 can transmit notifications to patients in a number of different ways. In certain embodiments, the notifications may be displayed on a “news feed” or RSS feed that displays notifications for all of the medical practitioners, or groups of medical practitioners, that a patient 101 is subscribed to or following. In certain embodiments, the notifications may be transmitted as messages to the patient's 101 inbox, or as emails, text messages, instant messages, automated phone calls or by other communication means. In certain embodiments, the patient 101 can select one or more ways of being notified. For example, if the patient 101 has an immediate need to book an appointment with a medical practitioner, the patient 101 can specify that a text message should be transmitted to the patient 101 in the case that a cancellation occurs and an appointment becomes available. In other cases, the patient may specify that certain types of notifications which are less important (e.g., profile changes made by a practitioner) should only be received via inbox messages or displayed on a news feed. Like any of the other notifications described herein, the subscribers notifications may include functions that permit a patient 101 to reserve an appointment, decline an appointment, cancel an appointment or to perform other types functions associated with the notification.

It should be noted that the features demonstrated on the interfaces in FIGS. 2-6, which permit a user to activate and customize the notifications that are being sent to patients 101, are merely provided as examples of how a user (e.g., medical practitioner) can customize the sending of notifications. However, the appointment scheduling platform 140 may provide a variety of additional customization features relating to sending notifications for appointment availabilities. For example, these customization features may permit a medical practitioner or other user to specify whether notifications should be sent automatically or in response to a user command (i.e., in response to a user explicitly indicating that notifications are to be sent). Likewise, the customization features may also permit the user to specify the type of notification that may be sent. For example, the customization features may allow the user to specify that notifications are to be sent by one or more communication channels such as text messages, inbox notifications, e-mail messages and automated telephone calls.

Even further, the customization features may permit a user to design or customize the content of the notification. For example, the user interfaces may permit a user to design the layout of a notification, the message content of a notification (e.g., which may indicate the time and location of the appointment), and the multimedia content (e.g., images, videos, animations, etc.) that is included in the notification. The interfaces may also include features that permit a user to incorporate particular types of functionalities into the message. For example, the user may incorporate a button or link into a notification, which may permit a user to automatically book or schedule an appointment by clicking on the button or link. The user may also be permitted to incorporate hyperlinks into the notification which may link to web pages that direct the user to a calendar 130 on the appointment scheduling platform 140 or which provide more information about the appointment, medical practitioner, or medical facility. In certain embodiments, templates may be provided to the user to assist the user with creating the notifications. Each template may be associated with, and may include, specific message content, multimedia content and/or functionality. Other types of customization features may also be provided.

As explained above, each of the notification features may include broadcast configuration options that permit a medical practitioner or other user to specify a subset of patients 101 that are to receive notifications for each of the notification features. For example, certain notification features may permit a medical practitioner to specify that notifications should only be transmitted to patients 101 having a particular illness or condition. Other types of configuration options may permit a medical practitioner to identify subsets of patients 101 based on a variety of other factors (e.g., based on age, gender, geographic location or other parameters). In some cases, the notification features may include broadcast configuration options that define a timeframe for identifying patients or appointments (e.g., the look-up interval feature 341, set interval feature 420 or timeframe feature 520). It should be recognized that any of the broadcast configuration options described above can be incorporated into any of the notification features described herein (e.g., the wait list feature 302, bump-up feature 303, interval follow-up feature 304, missed follow-up feature 301 or subscriber notification feature 305) and that other types of broadcast configuration options may also be utilized.

The broadcast configuration options permit a medical practitioner or other user to target specific subsets of patients that will or will not receive notifications. In certain embodiments, the patients 101 may also provide feedback which is utilized by the scheduling platform 140 to determine whether the patients 101 will receive notifications. For example, a patient 101 may be permitted to opt out of receiving notifications and this may prevent or limit the sending of notifications to the patient. As another example, patients may sign-up to receive certain types of notifications.

In certain embodiments, the broadcast configuration options may include a feature which is similar to the provider selection feature 220 described above. For example, the broadcast configuration options may include a scrollable list of patients. In order to identify or select a patient 101 for which notifications will be sent, a user may select (e.g., by clicking with a mouse or by tapping a touch screen) a patient 101 from the list and select an add button which will cause the name of the patient displayed in the list to appear in a window that displays all of the patients (or groups of patients) for which notifications will be sent. In order to remove a patient or group of patients from the subset of patients 101 that will receive notifications, the medical practitioner or other user may select a remove button as described above.

After a user has finished activating the notification features and customizing the broadcast configuration options associated with the activated notification features, the user may select the “Save & Publish” button (shown in FIG. 3) to finalize the campaign. Thereafter, notifications will be transmitted to patients in accordance with the selections made by the user.

FIG. 7 is an illustration 700 of a mobile device that is displaying an interface for scheduling appointments in accordance with certain embodiments of the present invention. The exemplary interface may be displayed to a patient 101 via a web browser or application that is installed on the patient's 101 mobile device (e.g., which may represent a client device 110). The interface provides a scrollable list of medical practitioners that indicates available appointments for each of the medical practitioners. In certain embodiments, the entries displayed on the interface may be related to the notification features described above. For example, if a patient 101 is signed up for a wait list or subscribed to a particular medical practitioner, a corresponding entry may appear in the list. Likewise, entries may also be provided in the list which identify bump-up appointments or which result from the patient failing to schedule a follow-up appointment.

A provider information section 720 is displayed for each provider in the list which includes the name of the medical practitioner, the type of services associated with the medical practitioner (e.g., dentist or dermatologist) and the address of the medical practitioner's office or medical practice. The provider information section 720 also includes a selectable telephone option 721 that enables a patient 101 to place a phone call using the mobile device to a telephone number associated with the medical practitioner. A provider location option 722 enables a patient 101 to view the location of the medical practitioner's office (or other place of employment) along with directions to the office. A list of appointment availabilities are also displayed for each of the medical practitioners. For each available appointment, a scheduling button 710 permits the patient to reserve or book an appointment.

A notification 750 is displayed on the interface to notify the patient 101 that a recommended appointment for a particular medical practitioner has become available. For example, the scheduling platform may indicate that the appointment is recommended because the patient 101 previously enrolled on a wait list pertaining to a particular practitioner and an appointment has become available for that practitioner. The exemplary notification 750 may be provided to patients 101 by the wait list feature 302, bump-up feature 303, interval follow-up feature 304, missed follow-up feature 301 or subscriber notification feature 305 as explained above.

The interface also includes a menu 730 that includes a plurality of selectable options or buttons. In certain embodiments, the menu 730 may include an appointment alert option that enables the patient to view notifications 750 or alerts regarding upcoming appointments and/or appointment availabilities that may be of interest to the patient 101. The menu 730 may also include an appointment listing option that enables the patient 101 to view all appointments that have been scheduled by the patient.

FIG. 8 is an illustration 800 of a mobile device that is displaying an interface associated with a subscriber notification feature in accordance with certain embodiments of the present invention. The exemplary interface may be displayed to a patient 101 via a web browser or application that is installed on the patient's 101 mobile device (e.g., which may represent a client device 110). The interface displays a scrollable list of medical practitioners who a patient 101 has subscribed to or is following (e.g., using the exemplary interface 600 shown in FIG. 6). In certain embodiments, the list of medical practitioners may be organized according to the type of medical practice (e.g., dermatologist, dentist or podiatrist) associated with the medical practitioners.

A provider entry 830 is included in the list for each medical practitioner who is subscribed to or being followed by the patient 101. Like the provider information section 720 illustrated in FIG. 7, a provider entry 830 includes the medical practitioner's name and address, along with a selectable telephone option 721 and selectable provider location option 722. An editing option 810 may be selected to enable a patient to stop following a particular medical practitioner 830 or to unsubscribe from a particular medical practitioner 830. In certain embodiments, the editing option 810 may also permit a patient 101 to adjust the settings associated with following a particular medical practitioner (e.g., adjust which notifications will be received for the medical practitioner). An add provider option 840 may be selected to enable a patient 101 to subscribe to or follow additional medical practitioners.

Each provider entry 830 further includes a subscriber indicator option 860 that displays the number of subscribers or followers that are associated with the medical practitioner and a provider schedule option 850 that indicates the number of appointments that have been scheduled with the medical practitioner and/or the number of patients that have scheduled an appointment with the medical practitioner. In certain embodiments, the provider schedule option 850 and the subscriber indicator option 860 may be selected by a patient 101. In response to selecting the provider schedule option 850, a schedule or calendar may be displayed which shows the appointments that have been reserved and/or available appointments for the medical practitioner. In response to selecting the subscriber indicator option 860, a list of the medical practitioner's subscribers or followers may be displayed.

FIGS. 9-13 illustrate exemplary methods associated with the notification features of the appointment scheduling platform 140 described above. According to certain embodiments, the methods described in these figures may be utilized in conjunction with the wait list notification feature 302, bump-up notification feature 303, interval follow-up feature 304, missed follow-up feature 301 and subscriber notification feature 305.

FIG. 9 is a flow chart of a method 900 for broadcasting wait list notifications in accordance with certain embodiments of the present invention. Patients 101 who desire appointments with medical practitioners or at medical facilities 160 during a time period when there are no available appointments are enrolled on a wait list (step 910). A patient 101 may accept an alternative appointment in a different time period which includes available appointments or may sign up for the wait list without accepting an alternative appointment. In some cases, the patients 101 may desire an appointment with specific practitioners or at specific medical facilities 160. Alternatively, the patients 101 may be willing to accept appointments with any medical practitioner or at any medical facility 160 during the time period.

An input is received which identifies a broadcast time for transmitting wait list notifications pertaining to appointment availabilities associated with the medical practitioners or medical facilities (step 920). As described above, interval selection features 210 and 212 may include a sliding bar or other input feature that allows an individual to specify a date and/or time prior to an appointment availability when the notifications are to be transmitted. For example, an individual may specify that wait list notifications are to be sent to patients 101 on a wait list two weeks prior to an appointment that had been cancelled or made available in order to notify the patients 101 about the newly available appointment. In certain embodiments, this step may be performed by a medical practitioner or other individual associated with a medical facility 160 by accessing a practitioner account provided by the platform 140.

Broadcast configuration settings are received that identify a subset of patients on the wait list (step 930). In certain embodiments, an individual may be presented with configuration options (e.g., options 331 and 332) that permit the individual to specify the patients 101 on the wait list who are to receive the wait list notifications. For example, an exemplary option 322 may permit the selection of all patients 101 who are associated with the appointment scheduling platform 140 (e.g., all patients that have signed up for accounts or memberships with the scheduling platform 140). Another exemplary option 331 may permit an individual to specify that only patients 101 on the wait list who have had prior visits with a particular doctor or at a particular medical facility 160 are to receive wait list notifications. The broadcast configuration options may further narrow the subset of selected patients based on the type of visit being sought. For example, if a cancelled appointment was originally for a heart condition, only patients on the wait list who are seeking an appointment for a heart condition may receive the wait list notifications. Other types of broadcast configuration options may also be provided to select the subset of patients on the wait list who are to receive the wait list notifications.

Next, appointments associated with the medical practitioners or medical facilities that become available within the desired time period are identified (step 940). As explained above, medical practitioners or other individuals may provide a schedule 130 on a scheduling server 120 that indicates appointment availabilities and scheduled appointments for a medical practitioner. Patients 101 may access the schedule 130 to reserve an appointment or to cancel an existing appointment. Thus, each appointment that becomes available during the time period in which an appointment is being sought by the patients on the wait list may be detected by the appointment scheduling platform 140.

Wait list notifications pertaining to the identified appointments may then be transmitted to the selected subset of patients at the specified broadcast time (step 950). In certain embodiments, the appointment scheduling platform 140 may periodically determine (e.g., twice a day) whether the broadcast time for a particular appointment cancellation has arrived and may send wait list notifications to the selected subset of patients in the case that the broadcast time has arrived. As explained above, the notifications may include content that identifies the available appointments and options that permit the patients 101 to reserve the appointments.

FIG. 10 is a flow chart of a method 1000 for broadcasting bump-up notifications in accordance with certain embodiments of the present invention. Initially, a schedule of patient appointments may be maintained (step 1010). A scheduling platform 140 accessible over a network 190 may store and provide access to schedules to patients 101, medical practitioners or other individuals. For example, patients 101 may interact with the schedules 140 to sign up for or cancel appointments, and the medical practitioners may update the schedules to indicate times when appointments are available or unavailable to be reserved. In certain embodiments, local schedules 170 stored on a client device 110 associated with a medical practitioner or medical facility 160 can be synced with a centralized schedule 140 provided by the scheduling platform 140.

A broadcast time is determined for transmitting bump-up notifications pertaining to appointment cancellations in the schedule (step 920). As described above, an interval selection feature may be utilized to specify the broadcast time for a broadcasting campaign. In certain embodiments, a medical practitioner or other individual associated with a medical facility 160 may specify the broadcast time when creating the campaign. Next, an appointment cancellation in the schedule is detected (step 1030). For example, a patient 101 may access the scheduling platform 140 via a patient account made accessible by the scheduling platform 140 in order to cancel an appointment. This cancellation may be detected by the scheduling platform 140.

After an appointment cancellation is detected, patients 101 who have appointments scheduled within a predetermined timeframe occurring after the appointment cancellation are identified (step 1040). For example, the look-up interval feature 341 described above permits the individual setting up a campaign to select a timeframe as part of the broadcast configuration options for the bump-up notification feature. Thus, the scheduling platform 140 may identify patients that have scheduled appointments falling within the time frame identified by the look-up interval feature 341.

Broadcast configuration options may then be utilized to select a subset of the identified patients having appointments within the timeframe (step 1050). For example, exemplary configuration options may include the interval feature 342 described above. The configuration options may permit a medical practitioner or other individual to narrow the subset of patients who are to receive bump-up notifications to consist of patients having appointments with the same visit duration (e.g., a half-hour or hour visit) as the appointment cancellation detected in the schedule. Likewise, the broadcast configuration options may also permit the individual to specify that bump-up notifications are only to be received by patients 101 having an appointment associated with the same or similar type of visit (e.g., a visit related to a heart condition, routine check-up, bone fracture or other medical condition) as the detected appointment cancellation. The options may permit the individual to select the subset of patients based on other types of criteria as well. For example, options may permit the selection of patients to be based on the location of a patient and/or medical practitioner, an age of patient, a patient's insurance provider or whether a patient had a history with a medical practitioner of medical facility 160 (e.g., attended a prior visit with a medical practitioner or medical facility 160).

The bump-up notifications may then be transmitted to the selected subset of patients 101 at the specified broadcast time (step 1060). For example, in the case that the specified broadcast time is set for one month, bump-up notifications may be transmitted one month prior to the date of the detected appointment cancellation to the subset of patients who satisfy the criteria of specified by the broadcast configuration options. As explained above, the notifications may include content that identifies the available appointments and options that permit the patients 101 to reserve the appointments.

FIG. 11 is a flow chart of a method 1100 for broadcasting interval follow-up notifications in accordance with certain embodiments of the present invention. Initially, the scheduling platform 140 may identify patients 101 who have not attended a follow-up appointment within a particular timeframe after an initial or prior appointment (step 1110). For example, a set interval feature 420 may be provided that permits a medical practitioner or other individual associated with a medical facility 160 to specify a timeframe. The scheduling platform 140 may look back in time and identify the patients 101 who have not attended an appointment in the specified timeframe. For example, if the set interval feature 420 is set for six months, the scheduling platform 150 may identify all patients who have not attended a follow-up appointment with a practitioner or facility within the past six months.

Broadcast configuration options are utilized to select a subset of the identified patients (who did not attend an appointment within the timeframe) to receive interval follow-up notifications (step 1120). In certain embodiments, exemplary patient selection options may include the age group feature 430 and diagnosis feature 440 discussed above. Other types of patient selection options may also be utilized to select the subset of patients 101.

Next, schedules associated with medical practitioners and/or medical facilities are analyzed to detect appointment availabilities (step 1130). As explained above, the scheduling platform 140 may store and provide access to schedules for medical practitioners or medical facilities 160. Thus, the scheduling platform 140 may detect availabilities in the schedules (possibly resulting from appointment cancellations).

Interval follow-up notifications may then be transmitted to the selected subset of patients 101 to notify the patients about the detected appointment availabilities (step 1140). As explained above, the notifications may include content that identifies the available appointments and options that permit the patients 101 to reserve the appointments.

FIG. 12 is a flow chart of a method 1200 for broadcasting missed follow-up notifications in accordance with certain embodiments of the present invention. Initially, a patient 101 who attended a medical visit with a medical practitioner or at a medical facility is identified (step 1210). In certain embodiments, a medical practitioner or other individual associated with a medical facility may identify a patient 101 by notifying the scheduling platform 140 that a patient has attended a medical visit after the medical visit has occurred. In other embodiments, the scheduling platform 140 may identify the patient by analyzing the schedule information for a patient or practitioner which is stored on the scheduling server 120. The patient 101 may be identified in other ways as well.

A determination is made as to whether the patient 101 has scheduled a follow-up appointment (step 1220). If the patient 101 has already scheduled a follow-up appointment, the method proceeds back to step 1210. Otherwise, a scheduling time is determined that indicates when a patient is expected to schedule a follow-up visit (step 1230). For example, in certain embodiments, a medical practitioner or other individual associated with a medical facility may access the scheduling platform 140 and specify a date and/or time when a patient 101 is expected to attend a follow-up visit. In certain embodiments, the scheduling platform 140 may also store information that indicates when a patient is expected to return for a follow-up visit based on the type of appointment that the patient 101 attended and this information may be utilized by the scheduling platform 140 to determine the scheduling time. For example, if the patient 101 attended an annual checkup, it may be presumed that the scheduling time for the patient would be approximately one year from the date of the medical visit. In certain embodiments, the scheduling time may represent a timeframe or period of time.

Next, a determination is made regarding whether the scheduling time for the patient 101 falls outside of a specified look-up timeframe (step 1240). For example, as explained above, the missed follow-up feature 301 may include a timeframe feature 520 that specifies a look-up timeframe that may span over a period of time that includes both the past and future. The scheduling platform will check to see whether the scheduling time (indicating when a patient 101 is expected to schedule a follow-up visit) falls within or overlaps the look-up time frame.

If it is determined that the scheduling time for the patient 101 falls outside of the look-up timeframe, then the method proceeds back to step 1210. On the other hand, if it is determined that the scheduling time associated with the patient falls within the look-up timeframe, then one or more appointment availabilities may be identified (step 1250). In certain embodiments, the scheduling platform 150 may only identify appointment availabilities for the medical practitioner that handled the initial medical visit or for the medical facility where the initial medical visit took place. In certain embodiments, the appointment availabilities may relate to availabilities that have resulted from appointment cancellations.

A missed follow-up notification may then be transmitted to the patient to notify the patient about the identified appointment availabilities (step 1260). The notification may permit the patient to reserve one or more of the appointment availabilities. The method may then proceed back to block 1210 and proceed in the same manner as described above.

FIG. 13 is a flow chart of a method 1300 for broadcasting subscriber notifications in accordance with certain embodiments of the present invention. A provider selection is received from a patient 101 identifying at least one medical practitioner (step 1310). The provider selection identifies the medical practitioners that the patient wishes to subscribe to or follow. In certain embodiments, after a patient 101 accesses a patient account associated with the scheduling platform 140, the patient may navigate to an interface that permits the patient 101 to specify the provider selection. For example, the interface 600 in FIG. 6 permits a patient 101 to select medical practitioners and/or groups of medical practitioners. Data specifying the provider selection may be stored on the scheduling server 120 and associated with the patient's 101 account.

Next, a notification selection may be received from the patient 101 that specifies one or more triggering events for sending subscriber notifications to the patient (step 1320). In certain embodiments, a notification selector feature 630 may permit a patient 101 to identify or select categories of notifications that a patient 101 is interested in receiving. For example, a notification selection may indicate that a patient 101 wants to receive notifications pertaining to appointment availabilities and/or cancellations (possibly within a particular time period specified by the patient), forum postings, profile changes or other events associated with the medical practitioners identified by the provider selection. In certain embodiments, a separate notification selection may be provided for each medical practitioner or group of medical practitioners identified by the provider selection, thus enabling the patient 101 to select different categories of notifications for each selected practitioner. In alternative embodiments, a single notification selection is provided that applies to all medical practitioners identified by the provider selection. Data specifying the notification selection may be stored on the scheduling server 120 and associated with the patient's 101 account.

An event associated with the at least one medical practitioner is detected (step 1330). All events associated with medical practitioners (and patients) may be detected by the scheduling platform 140. For example, the scheduling platform 140 may detect the occurrence of an event when an appointment becomes available (e.g., as a result of an appointment cancellation), when updated scheduling information is provided for a medical practitioner, when the scheduling platform 140 detects an availability in a time period specified by a patient 101 or when actions occur that affect the schedule for a medical practitioner. Other events may include forum postings made by practitioners, updates to a medical practitioner's profile information (e.g., address, phone number, work hours or other related profile information) and any interaction that a medical practitioner has with the scheduling platform 140.

A determination is made as to whether the event associated with the at least one medical practitioner is a triggering event for the medical provider associated with the event (step 1340). As explained above, the notification selection provided by the patient 101 specifies triggering events for a particular medical practitioner or group of medical practitioners. In certain embodiments, information stored on the scheduling server 120 which is indicative of the notification selection provided by the patient 101 may be accessed by the scheduling platform 140 to determine whether the event is a triggering event.

The method proceeds back to step 1330 if it is determined that the event associated with the at least one medical practitioner is not a triggering event. On the other hand, if it is determined that the event associated with the at least one medical practitioner is a triggering event, then a subscriber notification is transmitted to the patient which includes information related to the detected event (step 1350). For example, if the event is related to an appointment availability for the at least one medical practitioner, the subscriber notification may include information that notifies the patient 101 about the availability and may include a function that permits the patient to schedule an appointment for the time slot associated with the availability. As mentioned above, the subscriber notification may be provided to the patient in different ways (e.g., inbox notification, text message notification, e-mail notification, postal mail notification, automated phone call notification or other type of notification) and may permit the patient to perform different functions (e.g., reserve an appointment, cancel an existing appointment, view postings in a forum or blog or perform other functions related to the content of the notification).

While there have been shown and described and pointed out various novel features of the invention as applied to particular embodiments thereof, it should be understood that various omissions, substitutions and changes in the form and details of the systems and methods described and illustrated may be made by those skilled in the art without departing from the spirit of the invention. Amongst other things, the steps shown in the methods may be carried out in different orders in cases where such may be appropriate. Those skilled in the art will recognize that the particular hardware and devices that are part of the system described herein, and the general functionality provided by and incorporated therein, may vary in different embodiments of the invention. Accordingly, the particular system components shown in the figures are provided for illustrative purposes and to facilitate a full and complete understanding and appreciation of the various aspects and functionality of particular embodiments of the invention as realized in the system and method embodiments thereof. Those skilled in the art will appreciate that the invention can be practiced in other than the described embodiments, which are presented for purposes of illustration and not limitation.

What is claimed is:

1. A system for broadcasting notifications pertaining to appointment availabilities in schedules associated with medical practitioners, comprising:
a computing device having a processor and physical memory, the physical memory storing instructions that cause the processor to:
provide a scheduling platform adapted to transmit notifications identifying appointment availabilities in schedules for a plurality of medical practitioners, the scheduling platform including a first portion that is accessible to the medical practitioners and a second portion that is accessible to a plurality of patients;
provide the medical practitioners with access to a plurality of notification features via the second portion of the scheduling platform, wherein each of the notification features is configured to transmit notifications to a subset of patients in a separate broadcast scenario and each is associated with broadcast configuration settings that permit the medical practitioners to customize the respective broadcast scenarios;
receive a first set of selections from the medical practitioners pertaining to the notification features, the first set of selections activating notification features to enable transmission of the notifications according to corresponding broadcast scenarios associated with the activated notification features;
receive a second set of selections from the medical practitioners for customizing broadcast configuration settings for each of the activated notification features, the broadcast configuration settings at least identifying a subset of patients for receiving the notifications in accordance with the broadcast scenarios corresponding to the activated notification features;
periodically transmit the notifications to the subset of patients identified by the broadcast configuration settings; and
provide the subset of patients with access to the notifications via the first portion of the scheduling platform, wherein the notifications identify appointment availabilities in the schedules for the plurality of medical practitioners and permit the subset of patients to reserve the identified appointment availabilities in the schedules.

2. The system of claim 1, wherein the first set of selections include a selection that activates a wait list notification feature that is configured to transmit wait list notifications and processor is further configured to:
receive wait list enrollment requests from the patients via the first portion of the scheduling platform to enroll on one or more wait lists, wherein the wait list enrollment requests identify portions of the schedules for the medical practitioners in which there are no appointment availabilities or in which the patients are unable to attend any existing appointment availabilities;
identify appointment cancellations in the schedules for the medical practitioners;
determine whether the appointment cancellations occur within the portions of the schedules identified by the wait list enrollment requests; and
transmit the wait list notifications to the subset of patients identified by the broadcast configuration settings associated with the wait list notification feature in response to determining that the appointment cancellations occur within the portions of the schedules identified by the wait list enrollment requests.

3. The system of claim 1, wherein the first set of selections include a selection that activates a bump-up notification feature that is configured to transmit bump-up notifications and processor is further configured to:
receive appointment cancellations from patients via the first portion of the scheduling platform;
identify timeframes in the schedules occurring after the appointment cancellations;
select the subset of patients identified by the broadcast configuration settings associated with the bump-up notification feature, wherein selecting the subset of patients includes selecting patients that currently have reserved appointments in the timeframes occurring after the appointment cancellations; and
transmit the bump-up notifications to the selected subset of patients, the bump-up notifications including content that notifies the selected subset of patients that earlier appointments pertaining to the appointment cancellations are available to be reserved.

4. The system of claim 1, wherein the first set of selections include a selection that activates an interval follow-up notification feature that is configured to transmit interval follow-up notifications and processor is further configured to:
receive timeframe selections from the medical practitioners that identify periods of time;
receive diagnosis selections from the medical practitioners that identify medical conditions;
select the subset of patients identified by the broadcast configuration settings by identifying patients who have been diagnosed with the medical conditions identified by the diagnosis selections and who have not scheduled or attended medical visits within the periods of time identified by the timeframe selections;
analyze the schedules associated with the medical practitioners to detect the appointment availabilities; and
transmit the interval-follow notifications to the selected subset of patients, wherein the interval-follow notifications identify the detected appointment availabilities.

5. The system of claim 1, wherein the first set of selections include a selection that activates a missed follow-up notification feature that is configured to transmit missed follow-up notifications and processor is further configured to:
select the subset of patients identified by the broadcast configuration settings by identifying patients who previously attended medical visits and who failed to schedule or attend follow-up appointments within an expected timeframe occurring after the medical visits;
identify appointment availabilities in the schedules of the medical practitioners who rendered medical services during the previously attended medical visits;
transmit the missed follow-up notifications to the selected subset of patients, wherein the missed follow-up notifications identify the appointment availabilities in the schedules of the medical practitioners who rendered medical services during the previously attended medical visits.

6. The system of claim 1, wherein the first set of selections include a selection that activates a subscriber notification feature that is configured to transmit subscriber notifications and processor is further configured to:
receive subscriber requests from the patients that identify one or more of the medical practitioners, the subscriber requests being received via the first portion of the scheduling platform;
receive notification selections from the patients identifying categories of notifications;
select the subset of patients defined by the broadcast configuration settings based, at least in part, on the notification selections and subscriber requests;
transmit the subscriber notifications to the patients who submitted the subscriber requests, wherein the subscriber notifications include content for notifying the subset of patients about appointment availabilities in the schedules for the medical practitioners identified by the subscriber requests.

7. The system of claim 1, wherein the broadcast configuration settings are utilized to:
determine when the notifications are to be periodically transmitted;
select a timeframe in the schedules for identifying the subset of patients;
select the subset of patients based, at least in part, on visit durations or visit types; or
select the subset of patients based, at least in part, on age information or diagnosis information associated with the patients.

8. The system of claim 1, wherein the processor is further configured to:
transmit the notifications to the subset of patients via text messages or electronic mail.

9. The system of claim 1, wherein the notifications are periodically transmitted to inboxes associated with the patients and accessible to the patients via the first portion of the scheduling platform.

10. The system of claim 1, wherein the processor is further configured to:
receive requests from mobile devices associated with the patients to access the first portion of the scheduling platform; and
transmit data to the mobile devices that permits the patients to view the notifications and reserve appointment availabilities.

11. A method for broadcasting notifications pertaining to appointment availabilities in schedules associated with medical practitioners, comprising:
providing, with a processor, a scheduling platform adapted to transmit notifications identifying appointment availabilities in schedules for a plurality of medical practitioners, the scheduling platform including a first portion that is accessible to the medical practitioners and a second portion that is accessible to a plurality of patients;
providing the medical practitioners with access to a plurality of notification features via the second portion of the scheduling platform, wherein each of the notification features is configured to transmit notifications to a subset of patients in a separate broadcast scenario and each is associated with broadcast configuration settings that permit the medical practitioners to customize the respective broadcast scenarios;
receiving a first set of selections from the medical practitioners pertaining to the notification features, the first set of selections activating notification features to enable transmission of the notifications according to corresponding broadcast scenarios associated with the activated notification features;
receiving a second set of selections from the medical practitioners for customizing broadcast configuration settings for each of the activated notification features, the broadcast configuration settings at least identifying a subset of patients for receiving the notifications in accordance with the broadcast scenarios corresponding to the activated notification features;
storing the first set of selections and second set of selections on a non-transitory storage medium associated with the scheduling platform;
periodically transmitting, with a processor, the notifications to the subset of patients identified by the broadcast configuration settings; and
providing the subset of patients with access to the notifications via the first portion of the scheduling platform, wherein the notifications identify appointment availabilities in the schedules for the plurality of medical practitioners and permit the subset of patients to reserve the identified appointment availabilities in the schedules.

12. The method of claim 11, wherein the first set of selections include a selection that activates a wait list notification feature that is configured to transmit wait list notifications and the method further comprises:
receiving wait list enrollment requests from the patients via the first portion of the scheduling platform to enroll on one or more wait lists, wherein the wait list enrollment requests identify portions of the schedules for the medical practitioners in which there are no appointment availabilities or in which the patients are unable to attend any existing appointment availabilities;
identifying appointment cancellations in the schedules for the medical practitioners;
determining whether the appointment cancellations occur within the portions of the schedules identified by the wait list enrollment requests; and
transmitting the wait list notifications to the subset of patients identified by the broadcast configuration settings associated with the wait list notification feature in response to determining that the appointment cancellations occur within the portions of the schedules identified by the wait list enrollment requests.

13. The method of claim 11, wherein the first set of selections include a selection that activates a bump-up notification feature that is configured to transmit bump-up notifications and the method further comprises:
receiving the appointment cancellations from patients via the first portion of the scheduling platform;
identifying timeframes in the schedules occurring after the appointment cancellations;
selecting the subset of patients identified by the broadcast configuration settings associated with the bump-up notification feature, wherein selecting the subset of patients includes selecting patients that currently have reserved appointments in the timeframes occurring after the appointment cancellations; and
transmitting the bump-up notifications to the selected subset of patients, the bump-up notifications including content that notifies the selected subset of patients that earlier appointments pertaining to the appointment cancellations are available to be reserved.

14. The method of claim 11, wherein the first set of selections include a selection that activates an interval follow-up notification feature that is configured to transmit interval follow-up notifications and the method further comprises:
receiving timeframe selections from the medical practitioners that identify periods of time;
receiving diagnosis selections from the medical practitioners that identify medical conditions;
selecting the subset of patients identified by the broadcast configuration settings by identifying patients who have been diagnosed with the medical conditions identified by the diagnosis selections and who have not scheduled or attended medical visits within the periods of time identified by the timeframe selections;
analyzing the schedules associated with the medical practitioners to detect the appointment availabilities; and
transmitting the interval-follow notifications to the selected patients, wherein the interval-follow notifications identify the detected appointment availabilities.

15. The method of claim 11, wherein the first set of selections include a selection that activates a missed follow-up notification feature that is configured to transmit missed follow-up notifications and the method further comprises:
selecting the subset of patients identified by the broadcast configuration settings by identifying patients who previously attended medical visits and who failed to schedule or attend follow-up appointments within an expected timeframe occurring after the medical visits;
identifying appointment availabilities in the schedules of the medical practitioners who rendered medical services during the previously attended medical visits;
transmitting the missed follow-up notifications to the selected subset of patients, wherein the missed follow-up notifications identify the appointment availabilities in the schedules of the medical practitioners who rendered medical services during the previously attended medical visits.

16. The method of claim 11, wherein the first set of selections include a selection that activates a subscriber notification feature that is configured to transmit subscriber notifications and the method further comprises:
receiving subscriber requests from the patients which identify one or more of the medical practitioners, the subscriber requests being received via the first portion of the scheduling platform;
receiving notification selections from the patients identifying categories of notifications;
selecting the subset of patients defined by the broadcast configuration settings based, at least in part, on the notification selections and subscriber requests;
transmitting the subscriber notifications to the patients who submitted the subscriber requests, wherein the subscriber notifications include content for notifying the subset of patients about appointment availabilities in the schedules for the medical practitioners identified by the subscriber requests.

17. The method of claim 11, wherein the broadcast configuration settings are utilized to:
determine when the notifications are to be periodically transmitted;
select a timeframe in the schedules for identifying the subset of patients;
select the subset of patients based, at least in part, on visit durations or visit types; or
select the subset of patients based, at least in part, on age information or diagnosis information associated with the patients.

18. The method of claim 11, wherein the method further comprises:
transmitting the notifications to the subset of patients via text messages or electronic mail.

19. The method of claim 11, wherein the notifications are periodically transmitted to inboxes associated with the patients and accessible to the patients via the first portion of the scheduling platform.

20. The method of claim 11, wherein the method further comprises:
receiving requests from mobile devices associated with the patients to access the first portion of the scheduling platform; and
transmitting data to the mobile devices that permits the patients to view the notifications and reserve appointment availabilities.
